(19) United States
(12) Patent Application Publication
(10) Pub. No.: US 2004/0013615 A1
(43) Pub. Date: Jan. 22, 2004

(54) Pressurized hair treatment compositions comprising at least one amphiphilic linear diblock copolymer

(75) Inventors: Claude Dubief, Le Chesnay (FR); Franck Giroud, Clichy (FR)

Correspondence Address: STEPTOE & JOHSON LLP, 1330 Connecticut Avenue, NW, Washington, DC, 20036-1795, US

(73) Assignee: L'OREAL, Paris (FR)

(21) Appl. No.: 10/448,424

(22) Filed: May 30, 2003

Related U.S. Application Data
(60) Provisional application No. 60/385,590, filed on Jun. 5, 2002.

(30) Foreign Application Priority Data
May 31, 2002 (FR) 02 06730

(51) Int. Cl. A61K 9/00; A61K 7/06; A61K 7/11

(52) U.S. Cl. 424/47; 424/70.11; 424/70.16; 424/70.17

(57) ABSTRACT

The invention relates to hair compositions, packaged in an aerosol device, comprising, in a cosmetically acceptable medium,

at least one anionic or nonionic linear diblock block copolymer, consisting of a hydrophobic block and a hydrophilic block, with the exclusion of block copolymers of ethylene oxide and of propylene oxide, block copolymers containing urethane units and block copolymers containing siloxane units, and

at least one propellant,

and also to the use of these compositions for styling the hair.

The present invention relates to hair compositions, packaged in an aerosol device, comprising, in a cosmetically acceptable medium, at least one anionic or nonionic linear diblock block copolymer, consisting of a hydrophobic block and a hydrophilic block, and at least one propellant, and also to the use of these compositions for styling the hair.

Many block polymers are currently proposed for formulating compositions intended for styling, fixing and/or conditioning the hair.

Examples that may be mentioned include grafting polymers with a hydrocarbon-based skeleton bearing polydimethylsiloxane (PDMS) grafts, linear copolymers comprising blocks with a hydrocarbon-based skeleton and PDMS blocks, or polycondensates such as polyurethanes containing polyester and/or polyether and/or PDMS blocks.

Styling compositions containing cationic polyethylenic block copolymers are also known.

Thus, U.S. Pat. No. 4,030,512 describes styling lacquers or lotions containing cationic diblock or triblock copolymers comprising at least one block consisting of amine monomers. The highly cationic nature of these copolymers makes it difficult to remove them by washing and leads to an accumulation of the polymer on the surface of the hair. This results in an unpleasant and artificial feel after several applications.

U.S. Pat. No. 3,907,984 discloses hair fixing compositions containing cationic or nonionic diblock or triblock copolymers, which are insoluble in water but soluble in organic solvents such as ethanol. By virtue of their hydrophobic nature, these polymers give the hairstyle good moisture resistance. The cationic copolymers described in the said document have the problems of removal on washing mentioned above, while the nonionic copolymers described give mediocre cosmetic performance qualities. They have poor fixing power and have a tendency to make the hair dull. The blow-drying of hair treated with these copolymers is reflected by a powdery appearance, which obliges the user to wash his hair daily.

The Applicant has found that the use, in pressurized hair compositions, of a group of particular nonionic or anionic block copolymers, which are soluble or dispersible in the cosmetic medium used, leads to formulations that are readily sprayable, in particular when they contain a high proportion of water. These compositions apply easily to the hair and, surprisingly, give the appearance of dry hair much more quickly than the tested copolymers of the prior art. The nonionic or anionic block copolymers have satisfactory styling power and are readily removed by simple shampooing. The blow-drying of treated hair does not lead to a powdering effect, but leaves the hair soft and shiny.

A subject of the present invention is, consequently, a hair composition, packaged in an aerosol device, comprising, in a cosmetically acceptable medium,

at least one anionic or nonionic linear diblock block copolymer, consisting of a hydrophobic block and a hydrophilic block, with the exclusion of block copolymers of ethylene oxide and of propylene oxide, block copolymers containing urethane units and block copolymers containing siloxane units, and

at least one propellant.

A subject of the invention is also the use of such a hair composition for styling the hair.

The linear diblock block copolymers that may be used for the present invention are “amphiphilic” copolymers, i.e. copolymers comprising both a hydrophobic block and a hydrophilic block.

According to the present invention the term “hydrophobic block” means a block comprising at least 75 mol % of water-insoluble monomers, and the term “hydrophilic block” means a block comprising at least 75 mol % of water-soluble monomers.

The water-soluble monomers forming the hydrophilic block of the diblock copolymers used in the present invention may be of anionic or nonionic nature and may be used alone or in the form of a mixture containing two or more different monomers.

Examples of anionic water-soluble monomers that may be mentioned include ethylenically unsaturated carboxylic acids, such as acrylic acid, methacrylic acid, itaconic acid, fumaric acid, crotonic acid, maleic acid and maleic anhydride, 2-acrylamido-2-methylpropanesulphonic acid, styrenesulphonic acid, vinyl-sulphonic acid and vinylphosphonic acid.

The nonionic water-soluble monomers include, inter alia, acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinyl-formamide, N-methyl-N-vinylformamide, N-vinyllactams comprising a cyclic group of 4 to 9 carbon atoms, vinyl alcohol (copolymerized in the form of vinyl acetate and then hydrolysed), ethylene oxide, hydroxyethyl acrylate, hydroxypropyl acrylate, hydroxyethyl methacrylate and hydroxypropyl methacrylate.

The water-insoluble monomers forming the hydrophobic block of the diblock copolymers are preferably chosen from vinylaromatic monomers such as styrene and its alkyl derivatives, for instance 4-butylstyrene, α-methylstyrene and vinyltoluene, dienes such as butadiene and 1,3-hexadiene, and alkyl derivatives of dienes, such as isoprene and dimethylbutadiene, chloroprene, C₁₋₁₀ alkyl, C₆₋₁₀ aryl or C₆₋₁₀ aralkyl acrylates and C₁₋₁₀ alkyl, C₆₋₁₀ aryl or C₆₋₁₀ aralkyl methacrylates, for instance methyl, ethyl, n-butyl, 2-ethylhexyl, tert-butyl, isobornyl, phenyl or benzyl (meth)acrylates, vinyl acetate, the vinyl ethers of formula CH₂═CH—O—R and the allyl ethers of formula CH₂═CH—CH₂—O—R in which R represents a C₁₋₆ alkyl group, acrylonitrile, vinyl chloride, vinylidene chloride, caprolactone, ethylene, propylene, fluorovinyl monomers or vinyl monomers containing a perfluoro chain, such as fluoroalkyl acrylates and methacrylates or alkyl α-fluoroacrylates.

As indicated above with regard to the definition of the hydrophobic and hydrophilic blocks of the diblock copolymers, the water-insoluble monomers and the water-soluble monomers represent at least 75 mol % of the hydrophobic and hydrophilic blocks, respectively. In other words, the hydrophobic block may comprise up to 25 mol % of one or more water-soluble monomers. This proportion is preferably not more than 10 mol % and ideally less than or equal to 5 mol %.

Similarly, the hydrophilic block may comprise up to 25 mol %, preferably up to 10 mol % and ideally up to 5 mol %, of one or more water-insoluble monomers.

The linear diblock copolymers used obviously also include those in which the hydrophilic block and the hydrophobic block consist exclusively of water-soluble monomers and of water-insoluble monomers, respectively. These blocks may be homopolymer blocks or copolymer blocks containing two or more than two different monomers of the same type.

The number-average molecular mass of each block, whether it is hydrophobic or hydrophilic, and a copolymer or a homopolymer, is preferably between 500 and 100 000 and in particular between 500 and 50 000, with a polydispersity index (M_{w}/Mₙ) of between 1.01 and 3.0 and preferably between 1.1 and 2.5.

The weight ratio of the hydrophobic block to the hydrophilic block of the block copolymer is preferably between 1/20 and 20/1 and in particular between 1/10 and 10/1.

The diblock polymers of the invention may be prepared by the synthetic processes conventionally used for obtaining block polymers. Examples that may be mentioned include anionic or cationic polymerization, and controlled free-radical polymerization (see “New Method of Polymer Synthesis”, Blackie Academic & Professional, London, 1995, volume 2, page 1, or Trends Polym. Sci. 4, page 183 (1996) from C. J. Hawker), which may be used in various processes, for instance atom transfer radical polymerization (ATRP) (see JACS, 117, page 5614 (1995), from Matyjasezwski et al.), and the method with free radicals such as nitroxides (Georges et al., Macromolecules, 1993, 26, 2987).

These processes may also be used to obtain only one of the two types of blocks in the polymer of the invention, the other block being introduced into the final polymer by means of the initiator used, or alternatively via a coupling reaction between the hydrophilic and hydrophobic blocks.

The hair compositions of the present invention preferably contain the diblock block copolymers in dissolved or finely dispersed form in the cosmetically acceptable medium.

The block copolymers are consequently preferably soluble or dispersible in the cosmetically acceptable medium.

The expression “compounds that are soluble” in a given medium means compounds (monomers or polymers) which, when introduced into the said medium at 25° C., at a weight concentration equal to 0.5%, and optionally neutralized, allow the production of a macroscopically homogeneous and transparent solution, i.e. a solution having a light transmittance value, at a wavelength equal to 500 nm, through a sample 1 cm thick, of at least 70%, and preferably of at least 80%.

The expression “compounds that are dispersible” in a medium means compounds (monomers or polymers) which, when introduced into the said medium at 25° C. at a weight concentration equal to 0.5% by weight, allow the production of a non-transparent homogeneous dispersion.

According to the invention, the expression “cosmetically acceptable medium” means a medium consisting of water or of one or more cosmetically acceptable organic solvents, for instance C₁₋₄ lower alcohols, in particular ethanol, isopropanol, tert-butanol and n-butanol, and alkylene glycols, for instance propylene glycol, or alternatively a mixture of water and one or more cosmetically acceptable organic solvents.

The composition of the cosmetically acceptable medium and its proportion in the final hair composition are preferably such that the overall content of volatile organic compounds (VOC) in the hair composition is not more than 55% by weight.

The amount of amphiphilic linear diblock copolymer in the hair compositions of the present invention depends on a large number of parameters, among which mention may be made of the molecular mass of the diblock copolymers, the respective sizes of the hydrophilic and hydrophobic blocks, and in particular the desired cosmetic effect.

Satisfactory styling power is generally obtained with an amount of linear diblock copolymers of between 0.01% and 20% by weight and preferably between 0.1% and 15% by weight relative to the total weight of the hair composition.

The term “styling power” means the ability of a substance to facilitate the shaping of the hair and to hold this shape for a relatively long period.

The propellant may be any liquefiable gas usually used in aerosol devices, such as dimethyl ether, C₃₋₅ alkanes, 1,1-difluoroethane, mixtures of dimethyl ether and of C₃₋₅ alkanes, and mixtures of 1,1-difluoroethane and of dimethyl ether and/or of C₃₋₅ alkanes.

It is most particularly preferred to use dimethyl ether and C₃₋₅ alkanes as propellant, and in particular propane, n-butane and isobutane.

The liquid phase/propellant weight ratio in the pressurized hair compositions of the present invention is preferably between 50 and 0.05 and in particular between 25 and 1.5.

The hair compositions of the present invention may also contain one or more cosmetic ingredients or formulation ingredients usually used in cosmetics.

Examples of such ingredients that may be mentioned include anionic, cationic, nonionic or amphoteric film-forming polymers, other than the amphiphilic diblock copolymers described above, volatile or non-volatile silicones, anionic, cationic, amphoteric or nonionic surfactants, thickeners, nacreous agents, UV-screening agents, free-radical scavengers, fragrances, preserving agents, pigments, colorants, pH regulators, solubilizers, plasticizers, antifoams, waxes, oils, vitamins, conditioners and organic or mineral particles, which are synthetic or of natural origin.

Needless to say, a person skilled in the art will take care to select this or these optional additional additive(s) and/or the amounts thereof such that the intrinsic advantageous properties of the compositions of the invention are not adversely affected by the envisaged addition(s).

The hair compositions are preferably hair lacquers or mousses.

The invention is illustrated with the aid of the example that follows.

### EXAMPLE 1

Hair Lacquers

Five hair lacquers (Compositions A to E) with a volatile organic compounds content of 55% by weight, and each containing 10% by weight of fixing block copolymers, respectively, are prepared.

Compositions A and B according to the invention contain anionic and nonionic diblock copolymers, respectively, whereas the comparative compositions C and D contain cationic block copolymers and comparative composition E contains a multiblock polyurethane.

| | A | B | C | D | E |
|---|---|---|---|---|---|
| Anionic diblock | 10% | | | | |
| PS-b-PAA^{(a)} | | | | | |
| Nonionic diblock | | 10% | | | |
| PS-b-PHEMA^{(b)} | | | | | |
| Cationic diblock^{(c)} | | | 10% | | |
| Cationic diblock^{(d)} | | | | 10% | |
| Multiblock polyurethane^{(e)} | | | | | 10% |
| Fragrance | qs | qs | qs | qs | qs |
| Ethanol | 20% | 20% | 20% | 20% | 20% |
| AMP | pH 7 | pH 7 | pH 7 | pH 7 | pH 7 |
| DME | 35% | 35% | 35% | 35% | 35% |
| Water | qs | qs | qs | qs | qs |
| | 100% | 100% | 100% | 100% | 100% |

^{(a)}polystyrene (1 500 g/mol)-b-poly(acrylic acid)(44 000 g/mol) diblocks, sold by the company Polymer Source, Inc.
^{(b)}polystyrene (1 500 g/mol)-b-poly(hydroxyethyl methacrylate)(40 000 g/mol) diblocks, sold by the company Polymer Source, Inc.
^{(c)}polystyrene (18 600 g/mol)-poly(N-methyl-4-vinylpyridinium iodide)(131 300 g/mol) diblock, sold by the company Polymer Source, Inc.
^{(d)}poly(lauryl methacrylate-b-N,N-dimethylaminoethyl methacrylate) diblocks, cationic diblock polymer according to US 4 030 512
^{(e)}Luviset ® Pur, anionic multiblock polyurethane sold by the company BASF.

Each of the above compositions is applied using an aerosol device to 10 heads of medium-length (about 20 cm long) chestnut-brown European hair, at a rate of 10 grams per head of hair.

A group of 5 experts evaluates the fineness of the spray, the drying speed, the lacquering power and the ease of removal by washing using a grading scale ranging from 0 to 5.

The results, expressed in the form of an average of the grades attributed, are summarized in the table below.

| | A | B | | | |
|---|---|---|---|---|---|
| | according to | according to | | | |
| | the invention | the invention | C | D | E |
| Vaporization quality | 4.3 | 3.8 | 3.7 | 4.1 | 1.9 |
| Drying speed | 4.8 | 3.5 | 3.5 | 3.2 | 1.0 |
| Lacquering power | 3.6 | 3.9 | 4.1 | 4.3 | 3.5 |
| Removal by washing | 4.5 | 4.8 | 0.5 | 1.1 | 3.6 |

These results show that the cationic diblock polymers used in the prior art give cosmetic results that are comparable to those of the anionic or nonionic diblock polymers according to the invention, but have the drawback of being difficult to remove by washing, which is reflected by a relatively unnatural, laden feel after washing.

The results also show that the cosmetic compositions according to the invention dry faster and allow better spraying than a composition of the prior art (Composition E) containing an anionic amphiphilic multiblock polyurethane.

1. Hair composition, packaged in an aerosol device, comprising, in a cosmetically acceptable medium,
at least one anionic or nonionic linear diblock block copolymer, consisting of a hydrophobic block and a hydrophilic block, with the exclusion of block copolymers of ethylene oxide and of propylene oxide, block copolymers containing urethane units and block copolymers containing siloxane units, and
at least one propellant.

2. Hair composition according to claim 1, characterized in that the linear diblock block copolymer(s) are soluble or dispersible in the medium used.

3. Hair composition according to claim 1 or 2, characterized in that the hydrophilic block is formed from water-soluble monomers chosen from anionic water-soluble monomers and nonionic water-soluble monomers or a mixture thereof.

4. Hair composition according to claim 3, characterized in that the anionic water-soluble monomers are chosen from ethylenically unsaturated carboxylic acids, 2-acrylamido-2-methylpropanesulphonic acid, styrenesulphonic acid, vinylsulphonic acid and vinylphosphonic acid.

5. Hair composition according to claim 3, characterized in that the nonionic water-soluble monomers are chosen from acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate and hydroxypropyl methacrylate.

6. Hair composition according to any one of the preceding claims, characterized in that the hydrophobic block is formed from water-insoluble monomers chosen from vinylaromatic monomers, dienes and alkyl derivatives of dienes, chloroprene, C₁₋₁₀ alkyl, C₆₋₁₀ aryl or C₁₋₁₀ aralkyl acrylates, C₁₋₁₀ alkyl, C₆₋₁₀ aryl or C₁₋₁₀ aralkyl methacrylates, vinyl acetate, the vinyl ethers of formula CH₂═CH—O—R and the allyl ethers of formula CH₂═CH—CH₂—O—R in which R represents a C₁₋₆ alkyl group, acrylonitrile, vinyl chloride, vinylidene chloride, caprolactone, ethylene, propylene and fluorovinyl monomers or vinyl monomers containing a perfluoro chain.

7. Hair composition according to any one of the preceding claims, characterized in that the hydrophilic block contains up to 25 mol %, preferably up to 10 mol % and ideally up to 5 mol % of one or more water-insoluble monomers according to claim 6.

8. Hair composition according to any one of the preceding claims, characterized in that the hydrophobic block contains up to 25 mol %, preferably up to 10 mol % and ideally up to 5 mol % of one or more water-soluble monomers according to any one of claims 3 to 5.

9. Hair composition according to any one of the preceding claims, characterized in that the weight ratio of the hydrophobic block to the hydrophilic block in the diblock copolymer is between 1/20 and 20/1 and preferably between 1/10 and 10/1.

10. Hair composition according to any one of the preceding claims, characterized in that the linear diblock copolymer(s) is(are) present in a proportion of from 0.01% to 20% by weight and preferably in a proportion of from 0.1% to 15% by weight, relative to the total weight of the composition.

11. Hair composition according to any one of the preceding claims, characterized in that the propellant is chosen from dimethyl ether, C₃₋₅ alkanes, 1,1-difluoroethane, mixtures of dimethyl ether and of C₃₋₅ alkanes, and mixtures of 1,1-difluoroethane and of dimethyl ether and/or of C₃₋₅ alkanes.

12. Hair composition according to any one of the preceding claims, characterized in that it also contains cosmetic additives and/or formulation adjuvants such as anionic, cationic, nonionic or amphoteric film-forming polymers that are soluble or dispersible in an aqueous or aqueous-alcoholic medium, volatile or non-volatile silicones, anionic, cationic, amphoteric or nonionic surfactants, thickeners, nacreous agents, UV-screening agents, free-radical scavengers, fragrances, preserving agents, pigments, colorants, pH regulators, solubilizers, plasticizers, antifoams, waxes, oils, vitamins, conditioners and organic or mineral particles, which are synthetic or of natural origin.

13. Hair composition according to any one of the preceding claims, characterized in that it contains not more than 55% by weight of volatile organic compounds.

14. Use of a hair composition according to any one of the preceding claims, for styling the hair.
